(19) United States
(12) Patent Application Publication
(10) Pub. No.: US 2020/0229782 A1
(43) Pub. Date: Jul. 23, 2020

(54) Non-Destructive Inspection Method

(71) Applicant: Konica Minolta, Inc., Chiyoda-ku, Tokyo (JP)

(72) Inventors: Masahiro IMADA, Tokyo (JP); Takuji HATANO, Osaka (JP); Koujirou SEKINE, Osaka (JP); Masaaki TSUCHIDA, Tokyo (JP); Tsukasa YAGI, Hyogo (JP)

(73) Assignee: Konica Minolta, Inc., Chiyoda-ku, Tokyo (JP)

(21) Appl. No.: 16/647,107

(22) PCT Filed: Sep. 7, 2018
(86) PCT No.: PCT/JP2018/033165

§ 371 Date: Mar. 13, 2020

(30) Foreign Application Priority Data
Sep. 19, 2017 (JP) 2017-178497

(51) Int. Cl. A61B 6/00; G01N 23/083; G01N 23/223; G03B 42/02

(52) U.S. Cl. CPC A61B 6/484; G03B 42/025; G01N 23/223; G01N 23/083

(57) ABSTRACT

A non-destructive inspection method for inspecting an object to be inspected using a plurality of different types of non-destructive inspection means is shown. The method includes the following, fixedly forming common marks that can be detected by any of the plurality of non-destructive inspection means on the object to be inspected; then detecting the object to be inspected including the marks by the plurality of non-destructive inspection means respectively; and comparing the detection results by the plurality of non-destructive inspection means using the marks as positional references.

### TECHNICAL FIELD

The present invention relates to a non-destructive inspection method.

### BACKGROUND ART

There may be a case where an object to be inspected is desired to be inspected using a plurality of different types of non-destructive inspection means for example, a case where a lithium ion battery (hereinafter referred to as “LIB”) is inspected using an X-ray radiographing apparatus (X-ray Talbot radiographing apparatus or the like) and a magnetic field distribution measuring apparatus. It is possible to know, by X-ray radiographing, structural defects, presence or absence or location of a foreign substance in a LIB. It is also possible to visualize a current distribution in the LIB and know the magnitude of a leakage current and a leakage position by measuring a magnetic field distribution.

In actual inspections, it is necessary to make determinations not based on individual inspection results alone but based on multiple factors by combining both detection results. For example, even when a foreign substance is found by X-ray radiographing, if the foreign substance is not related to a leakage, there may be no problem. Alternatively, when a leakage occurs, it may be possible to grasp, by X-ray radiographing on the part in question, what the cause is and make a multiple-factor based determination by combining both detection results.

When multiple-factor based determinations are made based on the detection results by a plurality of different types of non-destructive inspection means, even when the respective detection results are individual ones, there may be a distinction between the front and back of a sample, orientation at the time of detection, the way how the object to be inspected is placed at the time of detection, displacement of coordinates for each apparatus (XY scale, orthogonality or the like), and they cannot be compared as they are. By simply marking with a magic marker or the like, the marks may not be imaged or reflected in the detection result, and if an attempt is made to compare and examine one detection result with the other detection result including a positional relationship, it may be necessary to calibrate the coordinates in advance so that the same positional relationship is obtained for each apparatus, form marks on the object to be inspected, pick up images of the marks using a camera separately at each time of inspection and calibrate the coordinates and the positional relationship and the like. In that case, there are problems that calibration is necessary, a camera or the like needs to be added, which causes the scale of the apparatus to increase.

Moreover, when the apparatus is laminated like a LIB, there is also a problem that even when a mark is placed on the outside of the package, the positional relationship with the internal electrodes or the like is shifted.

According to the invention described in Patent Literature 1, the one inspection apparatus identifies, prior to an inspection, the position of an object to be inspected based on two mutually orthogonal sides of an external line of an element formation region of a semiconductor substrate, which is an object to be inspected and external features such as orientation flats, and the other inspection apparatus identifies, prior to an inspection, the position of the object to be inspected based on positioning marks (not shown).

### CITATION LIST

### Patent Literature

[Patent Literature 1] JP 2915025B
[Patent Literature 2] JP 2017-104202A

### SUMMARY OF INVENTION

### Technical Problem

The invention described in Patent Literature 1 can adjust positions on the object to be inspected identified in the respective results of detection on a semiconductor substrate by a plurality of different types of non-destructive inspection means.

However, external features of the laminated object to be inspected such as a LIB are not stable and the outside shape may change between a previous inspection and a subsequent inspection. On the other hand, the invention described in Patent Literature 1 performs an optical search by capturing images using a camera prior to each inspection, which involves problems that a step of identifying a position before inspection is necessary and an addition of the camera or the like causes the scale of the apparatus to increase as described above.

The present invention has been made in view of the above problems with the prior arts, and it is an object of the present invention to simply and accurately adjust positions on an object to be inspected identified in detection results of each of a plurality of different types of non-destructive inspection means when inspecting the object to be inspected using the plurality of different types of non-destructive inspection means.

### Solution to Problem

An invention according to claim 1 to solve the above problems is a non-destructive inspection method for inspecting an object to be inspected using a plurality of different types of non-destructive inspection means, the method including:
fixedly forming common marks that can be detected by any of the plurality of non-destructive inspection means on the object to be inspected;
then detecting the object to be inspected including the marks by the plurality of non-destructive inspection means respectively; and
comparing the detection results by the plurality of non-destructive inspection means using the marks as positional references.

An invention according to claim 2 is the non-destructive inspection method according to claim 1, in which peculiar parts are identified based on the detection result by one of the plurality of non-destructive inspection means and the peculiar parts are then detected intensively by the other non-destructive inspection means.

An invention according to claim 3 is the non-destructive inspection method according to claim 1 or 2, in which information other than the positional references is recorded in the marks and the information is read from the detection results of the non-destructive inspection means.

An invention according to claim 4 is the non-destructive inspection method according to any one of claims 1 to 3, in which any two or more of X-ray radiographing means, magnetic field distribution measuring means, thermography radiographing means and hardness measuring means are included in the plurality of different types of non-destructive inspection means.

An invention according to claim 5 is the non-destructive inspection method according to claim 4, in which the plurality of different types of non-destructive inspection means include an X-ray Talbot radiographing apparatus as the X-ray radiographing means.

An invention according to claim 6 is the non-destructive inspection method according to any one of claims 1 to 5, in which the plurality of different types of non-destructive inspection means include the magnetic field distribution measuring means, and
raw materials constituting the marks include a magnetic substance.

An invention according to claim 7 is the non-destructive inspection method according to any one of claims 1 to 5, in which the plurality of different types of non-destructive inspection means include the X-ray radiographing means and the magnetic field distribution measuring means, and
raw materials constituting the marks include a substance with low X-ray transmission and a magnetic substance.

### Advantageous Effect of the Invention

According to the present invention, it is possible to simply and accurately adjust position's on an object to be inspected identified in detection results of each of a plurality of different types of non-destructive inspection means when inspecting the object to be inspected using the plurality of different types of non-destructive inspection means. It is thereby possible to make multiple-factor based determinations based on detection results by the plurality of different types of non-destructive inspection means.

### BRIEF DESCRIPTION OF THE DRAWINGS

FIG. 1A is a schematic view of an object to be inspected (or detection result thereof) in a mark formation stage of a non-destructive inspection method according to an embodiment of the present invention.

FIG. 1B is a schematic view of an object to be inspected (or detection result thereof) in an inspection A stage of the non-destructive inspection method according to the embodiment of the present invention.

FIG. 1C is a schematic view of an object to be inspected (or detection result thereof) in an inspection B stage of the non-destructive inspection method according to the embodiment of the present invention.

### DESCRIPTION OF EMBODIMENT

Hereinafter, an embodiment of the present invention will be described with reference to the accompanying drawings. The following description, however, is an embodiment of the present invention and not intended to limit the present invention.

An object to be inspected will be inspected using a plurality of different types of non-destructive inspection means. Examples of the plurality of different types of non-destructive inspection means include any two or more of X-ray radiographing means, magnetic field distribution measuring means, thermography radiographing means and hardness measuring means. In the present embodiment, a case will be described as an example, where an X-ray radiographing apparatus provided with X-ray radiographing means and a magnetic field distribution measuring apparatus provided With magnetic field distribution measuring means are used. The radiographing apparatus and the magnetic field distribution measuring apparatus are separate apparatuses and an object to be inspected moves between both apparatuses, and therefore the way the object to be inspected is placed with respect to origin coordinates of the respective apparatuses is not fixed.

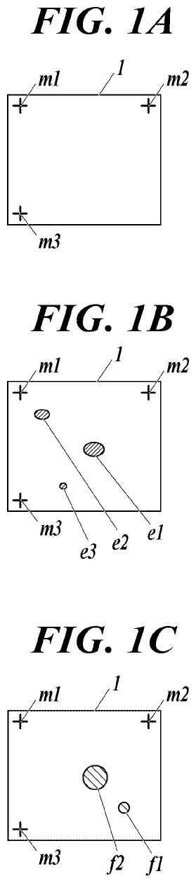

At a stage of manufacturing a product (e.g., the aforementioned LIB), which becomes an object to be inspected 1, marks m1, m2 and m3 that can be detected by an inspection A and an inspection B are fixedly formed by printing or the like on the object to be inspected 1 (FIG. 1A), the inspection A (FIG. 1B) is then performed, and the inspection B (FIG. 1C) is performed.

For example, the X-ray radiographing apparatus performs a detection as the inspection A and the magnetic field distribution measuring apparatus performs a detection as the inspection B. In this case, a substance with low X-ray transmission and a magnetic substance are applied as the raw materials for constituting the marks. For example, ink containing a heavy metal that hardly transmits X-rays and a magnetic substance is applied in printing of the marks. An X-ray Talbot radiographing apparatus (see Patent Literature 2) is applicable as the X-ray radiographing apparatus. The X-ray Talbot radiographing apparatus provides higher contrast than that in normal X-ray inspection, and it is thereby possible to regard the substance with low X-ray transmission and the magnetic substance as identical substances. However, in general, adopting different substances for the substance with low X-ray transmission and for the magnetic substance makes it possible to select the substances with high properties respectively and improve their respective detectabilities. Therefore, even when the X-ray Talbot radiographing apparatus is applied, different substances may be used for the substance with low X-ray transmission and the magnetic substance.

Note that an MR sensor, MI sensor, TMR sensor (tunnel-type magnetic resistance sensor) or the like is applicable as the magnetic sensor mounted on the magnetic field distribution measuring apparatus. A TMR sensor (tunnel-type magnetic resistance sensor) with higher sensitivity is preferably applicable.

Now, results of detection on the object to be inspected 1 including the marks m1, m2 and m3 are detected by the preceding inspection A, for example, as shown in FIG. 1B. As shown in FIG. 1B, it is assumed that peculiar parts e1, e2 and e3 are detected in the object to be inspected 1. Examples of the peculiar parts include an abnormal location, a location suspected to be an abnormal location, a location requiring additional inspection or the like.

Next, results of detection on the object to be inspected 1 including the marks m1, m2 and m3 are detected by the inspection B as shown, for example, in FIG. 1C. As shown in FIG. 1C, it is assumed that peculiar parts f1 and f2 are detected in the object to be inspected 1.

Next, the detection results of the inspections A and B are superimposed using the marks m1, m2 and m3, and compared and examined in a correct positional relationship. That is, the position detection results of the inspection A and the inspection B are compared using the marks m1, m2 and m3 as references. For example, when plane coordinate axes XY are defined using the marks m1, m2 and m3 as references and the coordinates on the coordinate axes XY are assumed to be (x1, y1), an abnormality is determined by contrasting, comparing and examining the detection values of the coordinates (x1, y1) in the detection result of the inspection A and the detection values of the coordinates (x1, y1) in the detection result of the inspection B. For example, in FIGS. 1B and 1C, since the peculiar part e1 and the peculiar part f2 have the same coordinates, they are determined as abnormal locations.

Based on the detection results of the inspections A and B, a more specific fault analysis may be made by a further inspection C (e.g., cross section TEM). At that time, locations to be analyzed may be selected using the marks m1, m2 and m3 as positional references.

As described above, it is possible to accurately grasp and compare the identical locations on the object to be inspected among a plurality of different types of inspections, and efficiently analyze causes for defects and perform shipment inspections.

Information other than positional references may be recorded in the marks m1, m2 and m3. For example, the marks m1, m2 and m3 are formed with one-dimensional or two-dimensional barcodes and individual identification numbers are recorded. Each inspection apparatus reads the above information from marks (code recording media) included in the detection results. The information reading is made possible by using the marks as common marks that can be detected by any non-destructive inspection means. As described above, since individual identification numbers integrally exist in the detection results, the respective detection results of an identical individual can be easily and reliably compared.

The non-destructive inspection means can be any means other than the X-ray radiographing means or the magnetic field distribution measuring means as long as it can measure an in-plane distribution non-destructively and obtain a detection image as a result. Examples of such non-destructive inspection means may include thermography radiographing means for measuring a thermal distribution by thermography, hardness measuring means for inspecting hardness using a probe needle for each coordinate. In the case of thermography, marks may be formed using an ink material whose emissivity is different from that of the surface of the object to be inspected or in the case of the probe needle, marks may be formed using an ink material whose hardness is different from that of the object to be inspected.

The present invention may be implemented using a method whereby peculiar locations are detected by the preceding inspection A and the peculiar locations found in the inspection A are finely and intensively inspected by the subsequent inspection B. That is, this is the method whereby peculiar parts are identified based on detection results by one of the plurality of non-destructive inspection means, and then the peculiar parts are intensively detected by the other non-destructive inspection means. “Intensively detecting peculiar parts” refers to regarding only peculiar parts as objects to be detected or detecting the peculiar parts with higher detection resolution than that of the remaining region.

It may be possible to consider, for example, a method whereby peculiar parts e1, e2 and e3 identified from results of detecting the whole surface of the object to be inspected by the X-ray radiographing apparatus in the inspection A are more finely measured by the magnetic field distribution measuring apparatus in the inspection B. X-ray radiographing can capture images of the whole surface at a time and detect the peculiar parts in a short time, whereas magnetic field distribution measurement adopts a scheme of performing measurement while scanning the measurement head, which takes more time if the area of the object to be inspected is wide. In such a case, the method of finely detecting only the peculiar parts found in X-ray radiographing using the magnetic field distribution measuring apparatus can shorten the inspection time.

As described above, when inspecting an object to be inspected using a plurality of different types of non-destructive inspection means, the non-destructive inspection method of the present embodiment can simply and accurately adjust positions on the object to be inspected identified in detection results of each of the plurality of non-destructive inspection means. It is thereby possible to make multiple-factor based determinations based on detection results by the plurality of different types of non-destructive inspection means.

In the above embodiment, the respective inspections are performed in order using different non-destructive inspection apparatuses, but even when a plurality of non-destructive inspection means detect the object to be inspected simultaneously or an identical composite apparatus provided with those means performs detections without moving the object to be inspected, it is possible to compare the detection results using marks included in the detection results as positional references. Therefore, even when the identical composite apparatus provided with the plurality of non-destructive inspection means is configured, it is possible to save time and trouble for calibrating coordinates of each non-destructive inspection means. Therefore, the present invention is not limited to a case where a plurality of different types of non-destructive inspection means are configured as separate apparatuses or a case where respective detections are performed in a time sequence.

### INDUSTRIAL APPLICABILITY

The present invention can be used for a non-destructive inspection method for a lithium ion battery or the like.

### REFERENCE SIGNS LIST

1 object to be inspected
e1, e2, e3 peculiar part
f1, f2 peculiar part
m1, m2, m3 mark

1. A non-destructive inspection method for inspecting an object to be inspected using a plurality of different types of non-destructive inspection means, the method comprising:
fixedly forming common marks that can be detected by any of the plurality of non-destructive inspection means on the object to be inspected;
then detecting the object to be inspected including the marks by the plurality of non-destructive inspection means respectively; and
comparing the detection results by the plurality of non-destructive inspection means using the marks as positional references.

2. The non-destructive inspection method according to claim 1, wherein peculiar parts are identified based on the detection result by one of the plurality of non-destructive inspection means and the peculiar parts are then detected intensively by the other non-destructive inspection means.

3. The non-destructive inspection method according to claim 1, wherein information other than the positional references is recorded in the marks and the information is read from the detection results of the non-destructive inspection means.

4. The non-destructive inspection method according to claim 1, wherein any two or more of X-ray radiographing means, magnetic field distribution measuring means, thermography radiographing means and hardness measuring means are included in the plurality of different types of non-destructive inspection means.

5. The non-destructive inspection method according to claim 4, wherein the plurality of different types of non-destructive inspection means comprise an X-ray Talbot radiographing apparatus as the X-ray radiographing means.

6. The non-destructive inspection method according to claim 1, wherein the plurality of different types of non-destructive inspection means comprise the magnetic field distribution measuring means, and
raw materials constituting the marks comprise a magnetic substance.

7. The non-destructive inspection method according to claim 1, wherein the plurality of different types of non-destructive inspection means comprise the X-ray radiographing means and the magnetic field distribution measuring means, and
raw materials constituting the marks comprise a substance with low X-ray transmission and a magnetic substance.
